SEQUENCE LISTING (48 sequences)

SEQ ID NO: 1 - DNA, 25 bp, synthetic construct
  misc_feature 1..25: note = Description of sequence: labeled oligionucleotide, name: Rh-L. vannamei-297F
  source 1..25: mol_type = other DNA
  modified_base 1: mod_base = OTHER; note = base labeled with fluorescent dye, dye name: 5' 6-FAM (Fluorescein)
  modified_base 25: mod_base = OTHER; note = base modified with propanal chain at the 3' end
  misc_feature 1..19: note = DNA
  misc_feature 20: note = RNA
  misc_feature 21..25: note = DNA
  misc_feature 1..25: note = molecule is DNA/RNA hybrid
gaatggttgaaagaggtgtcggaaa

SEQ ID NO: 2 - DNA, 26 bp, synthetic construct
  misc_feature 1..26: note = Description of sequence: labeled oligionucleotide, name: Rh-L. vannamei-388R
  source 1..26: mol_type = other DNA
  modified_base 1: mod_base = OTHER; note = base labeled with 5'-biotin
  modified_base 26: mod_base = OTHER; note = base modified with propanal chain at the 3' end
  misc_feature 1..20: note = DNA
  misc_feature 21: note = RNA
  misc_feature 21..26: note = DNA
  misc_feature 1..26: note = molecule is DNA/RNA hybrid
ccaagatctactgaagctccagcgtt

SEQ ID NO: 3 - DNA, 20 bp, synthetic construct
  misc_feature 1..20: note = Description of sequence: labeled oligionucleotide, name: FAM_IAC-2F
  source 1..20: mol_type = other DNA
  modified_base 1: mod_base = OTHER; note = base labeled with fluorescent dye, dye name: 5' 6-FAM (Fluorescein)
cgcaacgatgattattttct

SEQ ID NO: 4 - DNA, 20 bp, synthetic construct
  misc_feature 1..20: note = Description of sequence: labeled oligionucleotide, name: DIG_IAC-86R
  source 1..20: mol_type = other DNA
  modified_base 1: mod_base = OTHER; note = base labeled, label name: 5' Digoxigenin (NHS Ester)
  variation 3: note = n is a, c, g, or T
gtncctactattccagctca

SEQ ID NO: 5 - DNA, 26 bp, synthetic construct
  misc_feature 1..26: note = Description of sequence: labeled oligionucleotide, name: Rh.PM-2F
  source 1..26: mol_type = other DNA
  modified_base 1: mod_base = OTHER; note = base labeled with fluorescent dye, dye name: 5' 6-FAM (Fluorescein)
  modified_base 26: mod_base = OTHER; note = base modified with propanal chain at the 3' end
  misc_feature 1..20: note = DNA
  misc_feature 21: note = RNA
  misc_feature 21..26: note = DNA
  misc_feature 1..26: note = molecule is DNA/RNA hybrid
gggtttgggaattggytgctcccttg

SEQ ID NO: 6 - DNA, 26 bp, synthetic construct
  misc_feature 1..26: note = Description of sequence: labeled oligionucleotide, name: Rh.PM-2R
  source 1..26: mol_type = other DNA
  modified_base 1: mod_base = OTHER; note = base labeled with 5'-biotin
  modified_base 26: mod_base = OTHER; note = base modified with propanal chain at the 3' end
  misc_feature 1..20: note = DNA
  misc_feature 21: note = RNA
  misc_feature 21..26: note = DNA
  misc_feature 1..26: note = molecule is DNA/RNA hybrid
cagcagaagaccttaccccagccaag

SEQ ID NO: 7 - DNA, 19 bp, synthetic construct
  misc_feature 1..19: note = Description of sequence: labeled oligionucleotide, name: FAM_16SRna-F
  source 1..19: mol_type = other DNA
  modified_base 1: mod_base = OTHER; note = base labeled with fluorescent dye, dye name: 5' 6-FAM (Fluorescein)
cctcttgccatcggatgtg

SEQ ID NO: 8 - DNA, 21 bp, synthetic construct
  misc_feature 1..21: note = Description of sequence: labeled oligionucleotide, name: DIG_16SRna-R
  source 1..21: mol_type = other DNA
  modified_base 1: mod_base = OTHER; note = base labeled, label name: 5' Digoxigenin (NHS Ester)
ggctggtcatcctctcagacc

SEQ ID NO: 9 - DNA, 18 bp, synthetic construct
  misc_feature 1..18: note = Description of sequence: Primer, name: 83-F
  source 1..18: mol_type = other DNA
ggagccctactaggcgat

SEQ ID NO: 10 - DNA, 23 bp, synthetic construct
  misc_feature 1..23: note = Description of sequence: labeled oligionucleotide, name: 83-Rh-F
  source 1..23: mol_type = other DNA
  modified_base 23: mod_base = OTHER; note = base modified with propanal chain at the 3' end
  misc_feature 1..17: note = DNA
  misc_feature 18: note = RNA
  misc_feature 19..23: note = DNA
  misc_feature 1..23: note = molecule is DNA/RNA hybrid
ggagccctactaggcgatgatcc

SEQ ID NO: 11 - DNA, 21 bp, synthetic construct
  misc_feature 1..21: note = Description of sequence: Primer, name: 310-R
  source 1..21: mol_type = other DNA
gttcaaccagtaccggctcca

SEQ ID NO: 12 - DNA, 26 bp, synthetic construct
  misc_feature 1..26: note = Description of sequence: labeled oligionucleotide, name: 310-Rh-R
  source 1..26: mol_type = other DNA
  modified_base 26: mod_base = OTHER; note = base modified with propanal chain at the 3' end
  misc_feature 1..20: note = DNA
  misc_feature 21: note = RNA
  misc_feature 21..26: note = DNA
  misc_feature 1..26: note = molecule is DNA/RNA hybrid
gttcaaccagtaccggctccagctta

SEQ ID NO: 13 - DNA, 18 bp, synthetic construct
  misc_feature 1..18: note = Description of sequence: Primer, name: 179-F
  source 1..18: mol_type = other DNA
ggtttcgggaattggctt

SEQ ID NO: 14 - DNA, 24 bp, synthetic construct
  misc_feature 1..24: note = Description of sequence: labeled oligionucleotide, name: 179-Rh-F
  source 1..24: mol_type = other DNA
  modified_base 24: mod_base = OTHER; note = base modified with propanal chain at the 3' end
  misc_feature 1..17: note = DNA
  misc_feature 18: note = RNA
  misc_feature 19..24: note = DNA
  misc_feature 1..24: note = molecule is DNA/RNA hybrid
ggtttcgggaattggcttgtccaa

SEQ ID NO: 15 - DNA, 18 bp, synthetic construct
  misc_feature 1..18: note = Description of sequence: Primer, name: 304-R
  source 1..18: mol_type = other DNA
gttcccactcctctttcg

SEQ ID NO: 16 - DNA, 23 bp, synthetic construct
  misc_feature 1..23: note = Description of sequence: labeled oligionucleotide, name: 304-Rh-R
  source 1..23: mol_type = other DNA
  modified_base 23: mod_base = OTHER; note = base modified with propanal chain at the 3' end
  misc_feature 1..17: note = DNA
  misc_feature 18: note = RNA
  misc_feature 19..23: note = DNA
  misc_feature 1..23: note = molecule is DNA/RNA hybrid
gttcccactcctctttcgactac

SEQ ID NO: 17 - DNA, 20 bp, synthetic construct
  misc_feature 1..20: note = Description of sequence: Primer, name: 403-F
  source 1..20: mol_type = other DNA
ccttgctggggtttcttcaa

SEQ ID NO: 18 - DNA, 26 bp, synthetic construct
  misc_feature 1..26: note = Description of sequence: labeled oligionucleotide, name: 403-Rh-F
  source 1..26: mol_type = other DNA
  modified_base 26: mod_base = OTHER; note = base modified with propanal chain at the 3' end
  misc_feature 1..20: note = DNA
  misc_feature 21: note = RNA
  misc_feature 22..26: note = DNA
  misc_feature 1..26: note = molecule is DNA/RNA hybrid
ccttgctggggtttcttcaattttat

SEQ ID NO: 19 - DNA, 20 bp, synthetic construct
  misc_feature 1..20: note = Description of sequence: Primer, name: 564-R
  source 1..20: mol_type = other DNA
gctcccgctaagactggtaa

SEQ ID NO: 20 - DNA, 26 bp, synthetic construct
  misc_feature 1..26: note = Description of sequence: labeled oligionucleotide, name: 564-Rh-R
  source 1..26: mol_type = other DNA
  modified_base 26: mod_base = OTHER; note = base modified with propanal chain at the 3' end
  misc_feature 1..20: note = DNA
  misc_feature 21: note = RNA
  misc_feature 22..26: note = DNA
  misc_feature 1..26: note = molecule is DNA/RNA hybrid
gctcccgctaagactggtaaggatag

SEQ ID NO: 21 - DNA, 19 bp, synthetic construct
  misc_feature 1..19: note = Description of sequence: Primer, name: 478-F
  source 1..19: mol_type = other DNA
catgaaacctcctgccatt

SEQ ID NO: 22 - DNA, 24 bp, synthetic construct
  misc_feature 1..24: note = Description of sequence: labeled oligionucleotide, name: 478-Rh-F
  source 1..24: mol_type = other DNA
  modified_base 24: mod_base = OTHER; note = base modified with propanal chain at the 3' end
  misc_feature 1..18: note = DNA
  misc_feature 19: note = RNA
  misc_feature 20..24: note = DNA
  misc_feature 1..24: note = molecule is DNA/RNA hybrid
catgaaacctcctgccatttccct

SEQ ID NO: 23 - DNA, 21 bp, synthetic construct
  misc_feature 1..21: note = Description of sequence: Primer, name: 574-R
  source 1..21: mol_type = other DNA
agatttcggtctgtaagaagc

SEQ ID NO: 24 - DNA, 26 bp, synthetic construct
  misc_feature 1..26: note = Description of sequence: labeled oligionucleotide, name: 574-Rh-R
  source 1..26: mol_type = other DNA
  modified_base 26: mod_base = OTHER; note = base modified with propanal chain at the 3' end
  misc_feature 1..20: note = DNA
  misc_feature 21: note = RNA
  misc_feature 22..26: note = DNA
  misc_feature 1..26: note = molecule is DNA/RNA hybrid
agatttcggtctgtaagaagcattgc

SEQ ID NO: 25 - DNA, 19 bp, synthetic construct
  misc_feature 1..19: note = Description of sequence: Primer, name: 55-F
  source 1..19: mol_type = other DNA
gcttattcgagctgagctg

SEQ ID NO: 26 - DNA, 24 bp, synthetic construct
  misc_feature 1..24: note = Description of sequence: labeled oligionucleotide, name: 55-Rh-F
  source 1..24: mol_type = other DNA
  modified_base 24: mod_base = OTHER; note = base modified with propanal chain at the 3' end
  misc_feature 1..18: note = DNA
  misc_feature 19: note = RNA
  misc_feature 20..24: note = DNA
  misc_feature 1..24: note = molecule is DNA/RNA hybrid
gcttattcgagctgagctgagccg

SEQ ID NO: 27 - DNA, 18 bp, synthetic construct
  misc_feature 1..18: note = Description of sequence: Primer, name: 72-F
  source 1..18: mol_type = other DNA
tgagccaacctggggctt

SEQ ID NO: 28 - DNA, 23 bp, synthetic construct
  misc_feature 1..23: note = Description of sequence: labeled oligionucleotide, name: 72-Rh-F
  source 1..23: mol_type = other DNA
  modified_base 23: mod_base = OTHER; note = base modified with propanal chain at the 3' end
  misc_feature 1..17: note = DNA
  misc_feature 18: note = RNA
  misc_feature 19..23: note = DNA
  misc_feature 1..23: note = molecule is DNA/RNA hybrid
tgagccaacctggggctttgctg

SEQ ID NO: 29 - DNA, 21 bp, synthetic construct
  misc_feature 1..21: note = Description of sequence: Primer, name: 128-F
  source 1..21: mol_type = other DNA
gcgcatgcatttgtaataatc

SEQ ID NO: 30 - DNA, 26 bp, synthetic construct
  misc_feature 1..26: note = Description of sequence: labeled oligionucleotide, name: 128-Rh-F
  source 1..26: mol_type = other DNA
  modified_base 26: mod_base = OTHER; note = base modified with propanal chain at the 3' end
  misc_feature 1..20: note = DNA
  misc_feature 21: note = RNA
  misc_feature 22..26: note = DNA
  misc_feature 1..26: note = molecule is DNA/RNA hybrid
gcgcatgcatttgtaataatcttttg

SEQ ID NO: 31 - DNA, 18 bp, synthetic construct
  misc_feature 1..18: note = Description of sequence: Primer, name: 278-R
  source 1..18: mol_type = other DNA
cgaagctaagagaagcaa

SEQ ID NO: 32 - DNA, 23 bp, synthetic construct
  misc_feature 1..23: note = Description of sequence: labeled oligionucleotide, name: 278-Rh-R
  source 1..23: mol_type = other DNA
  modified_base 23: mod_base = OTHER; note = base modified with propanal chain at the 3' end
  misc_feature 1..17: note = DNA
  misc_feature 18: note = RNA
  misc_feature 19..23: note = DNA
  misc_feature 1..23: note = molecule is DNA/RNA hybrid
cgaagctaagagaagcaagaaat

SEQ ID NO: 33 - DNA, 18 bp, synthetic construct
  misc_feature 1..18: note = Description of sequence: Primer, name: 292-R
  source 1..18: mol_type = other DNA
gcttcaactccagacgaa

SEQ ID NO: 34 - DNA, 23 bp, synthetic construct
  misc_feature 1..23: note = Description of sequence: labeled oligionucleotide, name: 292-Rh-R
  source 1..23: mol_type = other DNA
  modified_base 23: mod_base = OTHER; note = base modified with propanal chain at the 3' end
  misc_feature 1..17: note = DNA
  misc_feature 18: note = RNA
  misc_feature 19..23: note = DNA
  misc_feature 1..23: note = molecule is DNA/RNA hybrid
gcttcaactccagacgaagctac

SEQ ID NO: 35 - DNA, 18 bp, synthetic construct
  misc_feature 1..18: note = Description of sequence: Primer, name: 304-R
  source 1..18: mol_type = other DNA
gtaccagcaccggcttca

SEQ ID NO: 36 - DNA, 23 bp, synthetic construct
  misc_feature 1..23: note = Description of sequence: labeled oligionucleotide, name: 304-Rh-R
  source 1..23: mol_type = other DNA
  modified_base 23: mod_base = OTHER; note = base modified with propanal chain at the 3' end
  misc_feature 1..17: note = DNA
  misc_feature 18: note = RNA
  misc_feature 19..23: note = DNA
  misc_feature 1..23: note = molecule is DNA/RNA hybrid
gtaccagcaccggcttcaactct

SEQ ID NO: 37 - DNA, 19 bp, synthetic construct
  misc_feature 1..19: note = Description of sequence: Primer, name: 29-F
  source 1..19: mol_type = other DNA
ggaatagtaggcacagcct

SEQ ID NO: 38 - DNA, 24 bp, synthetic construct
  misc_feature 1..24: note = Description of sequence: labeled oligionucleotide, name: 29-Rh-F
  source 1..24: mol_type = other DNA
  modified_base 24: mod_base = OTHER; note = base modified with propanal chain at the 3' end
  misc_feature 1..18: note = DNA
  misc_feature 19: note = RNA
  misc_feature 20..24: note = DNA
  misc_feature 1..24: note = molecule is DNA/RNA hybrid
ggaatagtaggcacagccttaaga

SEQ ID NO: 39 - DNA, 21 bp, synthetic construct
  misc_feature 1..21: note = Description of sequence: Primer, name: 227-F
  source 1..21: mol_type = other DNA
gcattcccccgaataaataat

SEQ ID NO: 40 - DNA, 26 bp, synthetic construct
  misc_feature 1..26: note = Description of sequence: labeled oligionucleotide, name: 227-Rh-F
  source 1..26: mol_type = other DNA
  modified_base 26: mod_base = OTHER; note = base modified with propanal chain at the 3' end
  misc_feature 1..20: note = DNA
  misc_feature 21: note = RNA
  misc_feature 22..26: note = DNA
  misc_feature 1..26: note = molecule is DNA/RNA hybrid
gcattcccccgaataaataatatgat

SEQ ID NO: 41 - DNA, 19 bp, synthetic construct
  misc_feature 1..19: note = Description of sequence: Primer, name: 358-F
  source 1..19: mol_type = other DNA
tgcacacgcaggagcttcc

SEQ ID NO: 42 - DNA, 24 bp, synthetic construct
  misc_feature 1..24: note = Description of sequence: labeled oligionucleotide, name: 358-Rh-F
  source 1..24: mol_type = other DNA
  modified_base 24: mod_base = OTHER; note = base modified with propanal chain at the 3' end
tgcacacgcaggagcttccgtcgt

SEQ ID NO: 43 - DNA, 19 bp, synthetic construct
  misc_feature 1..19: note = Description of sequence: Primer, name: 283-R
  source 1..19: mol_type = other DNA
acctgaggaggtaaggaga

SEQ ID NO: 44 - DNA, 24 bp, synthetic construct
  misc_feature 1..24: note = Description of sequence: labeled oligionucleotide, name: 283-Rh-R
  source 1..24: mol_type = other DNA
  modified_base 24: mod_base = OTHER; note = base modified with propanal chain at the 3' end
  misc_feature 1..18: note = DNA
  misc_feature 19: note = RNA
  misc_feature 20..24: note = DNA
  misc_feature 1..24: note = molecule is DNA/RNA hybrid
acctgaggaggtaaggagaagaat

SEQ ID NO: 45 - DNA, 19 bp, synthetic construct
  misc_feature 1..19: note = Description of sequence: Primer, name: 376-R
  source 1..19: mol_type = other DNA
aaagatggctaagtcgacg

SEQ ID NO: 46 - DNA, 24 bp, synthetic construct
  misc_feature 1..24: note = Description of sequence: labeled oligionucleotide, name: 376-Rh-R
  source 1..24: mol_type = other DNA
  modified_base 24: mod_base = OTHER; note = base modified with propanal chain at the 3' end
  misc_feature 1..18: note = DNA
  misc_feature 19: note = RNA
  misc_feature 20..24: note = DNA
  misc_feature 1..24: note = molecule is DNA/RNA hybrid
aaagatggctaagtcgacggaagt

SEQ ID NO: 47 - DNA, 21 bp, synthetic construct
  misc_feature 1..21: note = Description of sequence: Primer, name: 475-R
  source 1..21: mol_type = other DNA
ggtgtctgatactgggaaata

SEQ ID NO: 48 - DNA, 26 bp, synthetic construct
  misc_feature 1..26: note = Description of sequence: labeled oligionucleotide, name: 475-Rh-R
  source 1..26: mol_type = other DNA
ggtgtctgatactgggaaatagcgga

(19) United States
(12) Patent Application Publication
(10) Pub. No.: US 2025/0305066 A1
(43) Pub. Date: Oct. 2, 2025

(54) METHODS AND COMPOSITIONS USEFUL IN DISCRIMINATING BETWEEN SPECIES OF CERTAIN FISH AND SHELLFISH

(71) Applicant: The Florida State University Research Foundation, Inc., Tallahassee, FL (US)

(72) Inventor: Prashant Singh, Tallahassee, FL (US)

(21) Appl. No.: 19/092,325

(22) Filed: Mar. 27, 2025

Related U.S. Application Data
(60) Provisional application No. 63/570,329, filed on Mar. 27, 2024.

(51) Int. Cl. C12Q 1/6888; C12Q 1/6844

(52) U.S. Cl. CPC C12Q 1/6888; C12Q 1/6844; C12Q 2600/124; C12Q 2600/156

(57) ABSTRACT

Determining whether a particular sample is what it is labeled or sold as is an important concept, particularly to those who buy or sell food products. Particularly for end users, a fast, efficient, accurate way of determining whether a sample is being accurately marketed and sold is necessary. This invention allows a user to rapidly determine if a product is from a certain species or not, based on genetic markers for that product.

### CROSS-REFERENCE TO RELATED APPLICATIONS

This application claims benefit of U.S. Provisional Application No. 63/570,329, filed Mar. 27, 2024, incorporated herein by reference in its entirety.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Mar. 27, 2025, is entitled “10850-099US1_ST26.xml”, and is 112,204 bytes in size.

### BACKGROUND

The gold standard method for authenticating the identity of seafood species is the FDA's DNA barcoding technique, which targets the cytochrome C oxidase subunit I (COI) gene of the fish (Handy et al., 2011). Two barcoding methods are used for the identification of seafood specimens. The method targeting the ˜650 bp region is called barcoding and is applicable for fresh or minimally processed seafood specimens. Whereas the method targeting a smaller ˜100-300 bp region is referred to as mini-barcoding and is often useful for processed seafood specimens (Isaacs et al., 2020). The resulting sequence data for the sample is compared with FDA standard barcodes (Kress and Erickson, 2012). Sample sequence showing greater than 98% similarity with a standard barcode sequence is considered a positive match. The barcoding method is a robust method for seafood species identification and can identify up to 2749 species (FDA, 2011). However, as the process involves the overnight shipment of samples to a testing laboratory, DNA isolation, PCR amplification, sample clean up, sequencing of samples at a core facility, and data analysis the whole process can take up to five days. This lengthy testing time is a problem which limits the use of technology for seafood species identification.

Rapid seafood species-specific PCR-based tests that eliminate the need for DNA barcoding have been developed (Bayha et al., 2018; Lee et al., 2021; Wilwet et al., 2017; Isaacs et al., 2020). These assays commonly target the COI or 16S rRNA gene sequence for assay development. However, many species used for the substitution have a target gene sequence, which differs by only a few bases making the assay prone to false-positive results.

There is a need in the art for a low-cost, rapid assay to discern whether a certain sample is a same seafood species as mentioned on the label or not. Furthermore, there is a need for this method that can be performed onsite, at an in-house food processing facility, in a resource limited setting, using a minimally trained labor.

### SUMMARY

The present invention relates to a method of rapidly determining if a specific food product is present or not, the method comprising: providing a sample comprising at least one target sequence; placing the sample into at least one container; using reagents to amplify a sample; amplifying a sample using a small footprint nucleic acid amplification device, wherein the sample is amplified by exposing it to different sets and types of primers (conventional and rhPCR primers) in conditions suitable for nucleic acid amplification, where each set of primers comprises of a forward and reverse primers; exposing the amplified sequence to a means of detection, wherein the means of detection provides a present/not present result; and identifying whether the food or ingredient is present or not, based on the results.

Also disclosed is a kit can comprise a container for amplification of a sample; an instrument for sample collection; reagents for amplification of the sample; an instrument for rapid amplification of the sample; and a means of detecting whether the specific food product is present or not.

Further disclosed are nucleic acids with 90% or more identity to SEQ ID NOS: 1-48, or any combination thereof.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows an overall assay workflow standardized for the seafood testing.

FIG. 2A-2C shows PCR-Lateral Flow assay. FIG. 2A: PCR-lateral flow assay for the specific identification of white shrimp. FIG. 2B: Positive samples for PCR-lateral flow assay for the specific identification of white shrimp with an internal amplification control (IAC). FIG. 2C: Negative samples for PCR-lateral flow assay for the specific identification of white shrimp with an internal amplification control (IAC).

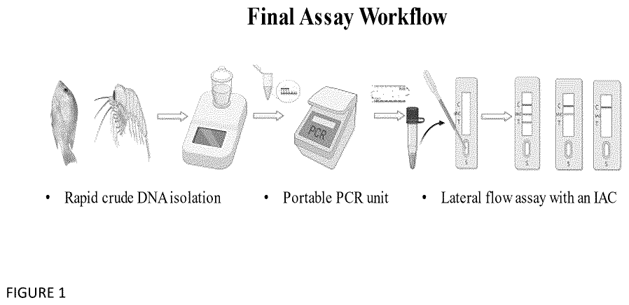

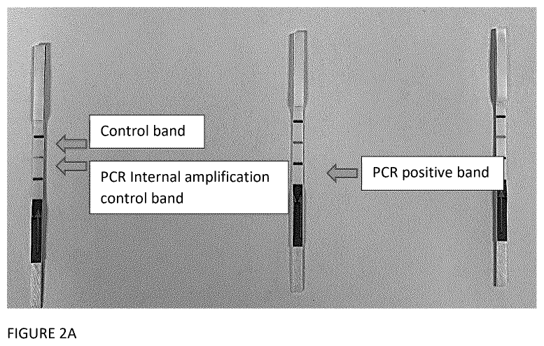

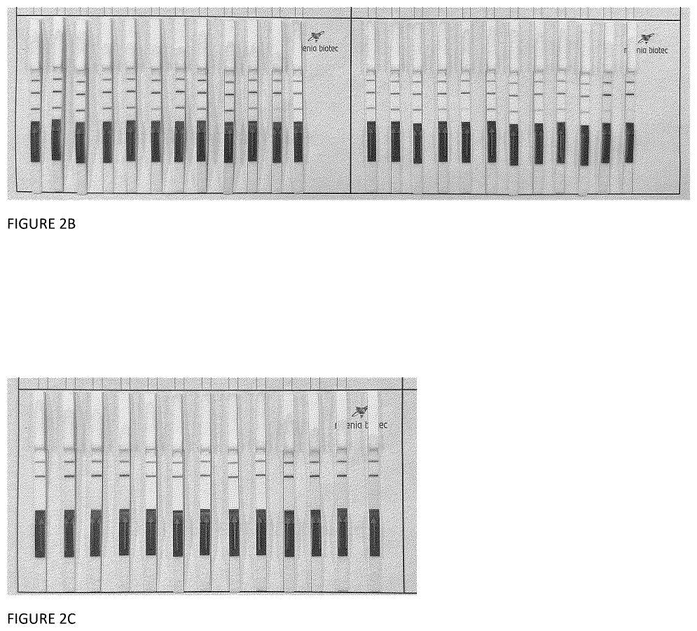

### DETAILED DESCRIPTION

### Definitions

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, “complementary” or “complementarity” refers to the ability of a nucleotide in a polynucleotide molecule to form a base pair with another nucleotide in a second polynucleotide molecule. For example, the sequence 5′-A-C-T-3′ is complementary to the sequence 3′-T-G-A-5′. Complementarity may be partial, in which only some of the nucleotides match according to base pairing, or complete, where all the nucleotides match according to base pairing. For purposes of the present invention “substantially complementary” refers to 90% or greater identity over the length of the target base pair region. The complementarity can also be 50, 60, 70, 75, 80, 85, 90, 91, 92, 93, 94, 95, 96, 97, 98, 99, or 100% complementary, or any amount below or in between these amounts.

As used herein, “nucleic acid sequence” refers to the order or sequence of nucleotides along a strand of nucleic acids. In some cases, the order of these nucleotides may determine the order of the amino acids along a corresponding polypeptide chain. The nucleic acid sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc. It may include modified bases, including locked nucleic acids, peptide nucleic acids and others known to those skilled in the art.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “primer” is a nucleic acid that contains a sequence complementary to a region of a template nucleic acid strand and that primes the synthesis of a strand complementary to the template (or a portion thereof). Primers are typically 18-20 base long, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified.

By “capture sequence,” which is also referred to herein as a “second nucleic acid sequence” is meant a sequence which hybridizes to the target nucleic acid and allows the first nucleic acid sequence, or primer sequence, to be in close proximity to the target region of the target nucleic acid.

A “target region” is a region of a target nucleic acid that is to be amplified, detected or both.

The “Tm” (melting temperature) of a nucleic acid duplex under specified conditions is the temperature at which half of the nucleic acid sequences are disassociated and half are associated. As used herein, “isolated Tm” refers to the individual melting temperature of either the first or second nucleic acid sequence in the cooperative nucleic acid when not in the cooperative pair. “Effective Tm” refers to the resulting melting temperature of either the first or second nucleic acid when linked together.

As used herein, “amplify, amplifying, amplifies, amplified, amplification” refers to the creation of one or more identical or complementary copies of the target DNA. The copies may be single stranded or double stranded. Amplification can be part of a number of processes such as extension of a primer, reverse transcription, polymerase chain reaction, isothermal polymerase chain reaction, nucleic acid sequencing, rolling circle amplification and the like.

As used herein, “purified” refers to a polynucleotide, for example a target nucleic acid sequence, that has been separated from cellular debris, for example, high molecular weight DNA, RNA and protein. This would include an isolated RNA sample that would be separated from cellular debris, including DNA. It can also mean non-native, or non-naturally occurring nucleic acid.

As used herein, “protein,” “peptide,” and “polypeptide” are used interchangeably to denote an amino acid polymer or a set of two or more interacting or bound amino acid polymers.

As used herein, “stringency” refers to the conditions, i.e., temperature, ionic strength, solvents, and the like, under which hybridization between polynucleotides occurs. Hybridization being the process that occurs between the primer and template DNA during the annealing step of the amplification process.

As used herein, “multiplex” refers to the use of PCR to amplify several different DNA targets (genes) simultaneously in a single assay or reaction. Multiplexing can amplify nucleic acid samples, such as genomic DNA, cDNA, RNA, etc., using multiple primers and any necessary reagents or components in a thermal cycler.

As used herein, a “sample” is from any source, including, but not limited to, a gas sample, a fluid sample, a solid sample, or any mixture thereof. In a preferred embodiment, the sample can be from shellfish, and can include, but is not limited to, shell, tissue, such as muscle or other flesh, or organs.

The term “sensitivity” refers to a measure of the proportion of actual positives which are correctly identified as such.

The term “confidence level” refers to the likelihood, expressed as a percentage, that the results of a test are real and repeatable, and not random. Confidence levels are used to indicate the reliability of an estimate and can be calculated by a variety of methods.

In certain embodiments, sequences of the present invention, including primer sequences, target sequences and IAC sequences may be identical to the sequences provided here in or comprise less than 100% sequence identity to the sequences provided herein. For instance, primer sequences, target sequences or IAC sequences of the present invention may comprise 90-100% identity to the sequences provided herein.

The terms “identical” or “percent identity,” in the context of two or more nucleic acids or sequences, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site found at ncbi.nlm.nih.gov/BLAST/or the like). Such sequences are then referred to as “substantially identical.” This definition also refers to, or applies to, the compliment of a particular sequence. The definition may also include sequences that have deletions, additions, and/or substitutions. To compensate for gene sequence diversity and to target multiple gene variants of the same genes, degenerated primer pairs (1-2 bases or approximately 5-10% alterations) are allowed.

As used herein, the term “nucleic acid” refers to a single or double-stranded polymer of deoxyribonucleotide bases or ribonucleotide bases read from the 5′ to the 3′ end, which may include genomic DNA, target sequences, primer sequences, or the like. In accordance with the invention, a “nucleic acid” may refer to any DNA or nucleic acid to be used in an assay as described herein, which may be isolated or extracted from a biological sample. The term “nucleotide sequence” or “nucleic acid sequence” refers to both the sense and antisense strands of a nucleic acid as either individual single strands or in the duplex. The terms “nucleic acid segment,” “nucleotide sequence segment,” or more generally, “segment,” will be understood by those in the art as a functional term that includes genomic sequences, target sequences, operon sequences, and smaller engineered nucleotide sequences that express or may be adapted to express, proteins, polypeptides or peptides. The nomenclature used herein is that required by Title 37 of the United States Code of Federal Regulations § 1.822 and set forth in the tables in WIPO Standard ST.25 (1998), Appendix 2, Tables 1 and 3.

The term “gene” refers to components that comprise bacterial DNA or RNA, cDNA, artificial bacterial DNA polynucleotide, or other DNA that encodes a bacterial peptide, bacterial polypeptide, bacterial protein, or bacterial RNA transcript molecule, introns and/or exons where appropriate, and the genetic elements that may flank the coding sequence that are involved in the regulation of expression, such as, promoter regions, 5′ leader regions, 3′ untranslated region that may exist as native genes or transgenes in a bacterial genome. The gene or a fragment thereof can be subjected to polynucleotide sequencing methods that determines the order of the nucleotides that comprise the gene. Polynucleotides as described herein may be complementary to all or a portion of a bacterial gene sequence, including a promoter, coding sequence, 5′ untranslated region, and 3′ untranslated region. Nucleotides may be referred to by their commonly accepted single-letter codes.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps. Similarly, any cell that “comprises,” “has” or “includes” one or more traits is not limited to possessing only those one or more traits and covers other unlisted traits.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular electrode is disclosed and discussed and a number of modifications that can be made to the electrode are discussed, specifically contemplated is each and every combination and permutation of the electrode and the modifications that are possible unless specifically indicated to the contrary. Thus, if a class of electrodes A, B, and C are disclosed as well as a class of electrodes D, E, and F and an example of a combination electrode, or, for example, a combination electrode comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures that can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to the arrangement of steps or operational flow; plain meaning derived from grammatical organization or punctuation; and the number or type of embodiments described in the specification.

### General Description

Seafood is a substantial source of nutrients such as proteins, polyunsaturated fatty acids, vitamin A, iodine, zinc, calcium, iron, and ω-3 fatty acids, making them an important component of the human diet (Aaker et al., 2020, Liu et al., 2021). Worldwide, fish and fish products contribute to about 20% of the per capita animal protein intake (FAO, 2018). Between 2008 and 2018, the global seafood consumption rate increased by about 19%, from 16.7 kg per capita to 20.5 kg per capita (FAO, 2020b). In the United States (U.S.), fresh and frozen finfish consumption is about 3.8 kg (8.3 pounds) per capita (NMFS, 2022).

The American Scallop (Placopecten magellanicus) is often substituted with scallops from other countries (e.g., China, Japan, Peru). Atlantic sea scallops are found in the Northwest Atlantic Ocean, from Newfoundland to Cape Hatteras, North Carolina. However, they can be substituted for not only scallops from other countries, but can also be substituted for other fish products which are not in the scallop family.

A study conducted by Oceana (2014 Report, available on Oceana's website) revealed misrepresentation of shrimp across the United States. DNA testing confirmed that 30 percent of the 143 shrimp products tested from 111 grocery stores and restaurants were misrepresented. Oceana also found that consumers are often provided with little information about the shrimp they purchase, including where and how it was caught or farmed, making it difficult, if not impossible, for them to make informed choices.

Oceana found misrepresented shrimp everywhere it tested, including rates of 43 percent in New York, NY, 33 percent in Washington, D.C., 30 percent in the Gulf of Mexico region (Pensacola and Fort Walton Beach, FL; Mobile and Orange Beach, AL; Biloxi and Ocean Springs, MS; New Orleans and LA, Louisiana; and Houston and Galveston, TX) and 5 percent in Portland, OR. Overall, 35 percent of the 111 vendors visited nationwide sold misrepresented shrimp. Of the 70 restaurants visited, 31 percent sold misrepresented shrimp, and 41 percent of the 41 grocery stores visited sold misrepresented products. Shrimp purchased from grocery stores and restaurants were misrepresented at the same rate-30 percent.

Rapid seafood species-specific PCR-based tests that eliminate the need for DNA barcoding have been developed (Bayha et al., 2018; Lee et al., 2021; Wilwet et al., 2017; Isaacs et al., 2020). These assays commonly target the COI or 16S rRNA gene sequence for assay development. However, many species used for the substitution have a target gene sequence, which differs by only a few bases making the assay prone to false-positive results. These limitations of PCR assay can be overcome using RNase H2-dependent PCR (rhPCR) approach (Dobosy et al., 2011). The rhPCR approach relies on the use of blocked primers and RNase H2 enzyme isolated from Pyrococcus abyssi (Will, 1992). RNase H2 enzyme enables the PCR assay to make a sequence-specific cut and activates the blocked primer only in the presence of the target DNA sequence. The rhPCR assay is highly specific toward their targets and can be performed using a conventional PCR instrument. Then those rhPCR amplicons can be detected using a lateral-flow kit. Therefore, the aim of this study was to standardize a highly specific rhPCR-coupled lateral flow assay, which can be used for the onsite identification of shrimp and scallop samples in a resource-limited setting.

### Methods of Detection

Disclosed herein is a method of rapidly determining if a specific food product is present or not, the method comprising: providing a sample comprising at least one target sequence; placing the sample into at least one container; using reagents to amplify a sample; amplifying a sample using a small footprint amplification device, wherein the sample is amplified by exposing it to different sets of primers in conditions suitable for nucleic acid amplification, where each set of primers comprises of a forward and reverse primer; exposing the amplified sequence to a means of detection, wherein the means of detection provides a present/not present result; and identifying whether the food product or ingredient is present or not based on the results.

In a specific example, the imported Pacific white shrimp (Litopenaeus vannamei) is commonly used to replace the domestic shrimp species (Litopenaeus setiferus) harvested from the United States water. Similarly, the American Scallop (Placopecten magellanicus) is often substituted with scallops from other countries (e.g., China, Japan, Peru). These can all be discriminated between using the assays disclosed herein.

The sample can be obtained from a variety of means. Typically, either a shellfish can be sampled, or a piece of meat or other tissue sample can be used. A toothpick, tweezers, a swab, or other small device for gathering a DNA sample can be used. The device can be passed along the surface of the sample, or can be plunged into the sample. The device for gathering the sample can then be placed in a tube, where it can be processed through a DNA extraction procedures, and centrifuged if needed, although this is not a necessary step and samples can be directly processed using a inhibitor-resistant mater mix (i.e., Platinum Direct PCR Universal Master Mix, KAPA PROBE Force). In one embodiment, a commercially available kit, such as PrepMan® Ultra Sample Preparation Reagent (Applied Biosystems, Life Technologies) or Extracta DNA Prep for PCR (Quanta Biosciences, Beverly, MA, USA) may be used to isolate DNA. According to one embodiment, suspended food particles may be separated from the media, for instance through filtration or centrifugation of the enriched sample, for example at 3,000-10,000×g. The cell pellet can be heat treated at 95° C. for 10 to 30 minutes (depending upon the samples), samples can be centrifuged and obtained supernatant containing crude DNA extract can be used as a sample for analysis described herein. The sample can then be exposed to amplification reagents (known to those of skill in the art) and amplified.

Amplification can occur by using a variety of devices. In a preferred embodiment, the amplification device can be small footprint, portable device such as a Watson PCR machine. In some embodiments, the machine can weigh less than 5, 10, 15, or 20 lbs, and can be less than 12″×12″, 15″×15″, 18″×18″, or 24″×24″.

Once amplified, the sample can be placed on a detection device, such as a lateral flow assay (LFA). Such lateral flow assays for the detection of a sample are known to those of skill in the art. LFAs are typically composed of a nitrocellulose membrane, sample pad, conjugate pad, wicking or absorbent pad, and backing pad. Nitrocellulose membranes are most commonly used as they facilitate a support capable of use for both reaction and detection, with capture biomolecules e.g., antibodies, are deposited on the nitrocellulose to form the test and control lines via a combination of electrostatic interactions, hydrogen bonds and/or hydrophobic interactions (Jauset-Rubio et al., 2016), herein incorporated in its entirety for its teaching concerning lateral flow assays). There are a large number of paper analytical devices (PAD) that have been developed for detection of PCR products using lateral flow assays. There are two main types of lateral flow nucleic acid tests, referred to as Nucleic Acid Lateral Flow (NALF) and Nucleic Acid Lateral Flow ImmunoAssay (NALFIA); NALF directly detects DNA exploiting capture and labeled reporter oligonucleotide probes, whereas NALFIA detects hapten-labeled DNA using capture and labeled reporter antibodies or streptavidin. Again, one of skill in the art can readily envision such assays for use with the present invention.

Importantly, the lateral flow assay can simply provide a “present/not present” result so that one skilled in the art can readily determine if the sample is a certain species or not. For example, if one were testing for the presence of red grouper, one would obtain a sample of meat, take a sample and amplify it, then expose the amplification product to a lateral flow assay designed to detect the presence of nucleic acid for red grouper. If red grouper is present, the assay will indicate a “positive.” Conversely, if red grouper is absent, the test will be negative, and no positive result will appear. In one embodiment, the lateral flow assay can comprise a control line to determine if the lateral flow strip and buffer are working or not. In one embodiment, the lateral flow assay can comprise an internal amplification control line to determine if PCR reactions are working or not. Again, one of skill in the art can readily determine how to design a lateral flow assay for use with this invention.

In some examples, a probe can be used to detect the target nucleic acid can be any probe known to those of skill in the art used in nucleic acid detection. The probe can be a single probe or a dual-labeled probe, such as those found in FRET systems. Detectable labels may include, but are not limited to, radiolabels, fluorochromes, including fluorescein isothiocyanate (FITC), biotin, digoxigenin, rhodamine, Texas Red, phycoerythrin, allophycocyanin, 6-carboxyfluorescein (6-FAM), 2′, 7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxy fluorescein (5-FAM) or N,N,N′,N′-tetramethyl-6-carboxyrho-damine (TAMRA); radioactive labels such as 32P, 35S, and 3H), and the like. In some embodiments, a detectable label may involve multiple steps (e.g., biotin-avidin, hapten-anti-hapten antibody, and the like). A primer useful in accordance with the invention may be identical to a particular target nucleic acid sequence and different from other sequences.

The probes selected and/or utilized by the methodologies of the invention can provide sensitivity and/or specificity of more than 90%, 95%, 96%, 97%, 98%, 99% or 100%. In some embodiments, sensitivity and specificity depends on the hybridization signal strength, number of probes used, the number of potential cross-hybridization reactions, the signal strength of the mismatch probes, if present, background noise, or combinations thereof.

The oligonucleotide probes can each be from about 5 to about 100 nucleotides, from about 10 to about 50 nucleotides, from about 15 to about 35 nucleotides. or from about 20 to about 30 nucleotides. In some embodiments, the probes are at least 5-mers, 6-mers, 7-mers, 8-mers, 9-mers, 10-mers, 11-mers, 12-mers, 13-mers, 14-mers, 15-mers, 16-mers, 17-mers, 18-mers. 19-mers, 20-mers, 21-mers, 22-mers, 23-mers, 24-mers, 25-mers, 26-mers, 27-mers, 28-mers, 29-mers. 30-mers, 31-mers, 32-mers, 33-mers, 34-mers, 35-mers, 36-mers, 37-mers, 38-mers, 39-mers, 40-mers. 41-mers, 42-mers, 43-mers, 44-mers, 45-mers, 46-mers, 47-mers, 48-mers, 49-mers, 50-mers, 51-mers 52-mers, 53-mers, 54-mers, 55-mers, 56-mers, 57-mers, 58-mers, 59-mers, 60-mers, 61-mers, 62-mers. 63-mers, 64-mers, 65-mers, 66-mers, 67-mers, 68-mers, 69-mers, 70-mers, 71-mers, 72-mers, 73-mers. 74-mers, 75-mers, 76-mers, 77-mers, 78-mers, 79-mers, 80-mers, 81-mers, 82-mers, 83-mers, 84-mers, 85-mers, 86-mers, 87-mers, 88-mers, 89-mers, 90-mers, 91-mers, 92-mers, 93-mers, 94-mers, 95-mers, 96-mers, 97-mers, 98-mers, 99-mers, 100-mers or combinations thereof

The amplification reaction described above needs reagents in order for amplification to occur. One of skill in the art can readily determine which reagents should be present in order to amplify a sample. Such reagents include, but are not limited to, PCR “Mastermix”; Taq polymerase; RNase H2 enzyme, RNase H2 enzyme buffer, and primers or labeled primers. Methods such as polymerase chain reaction (PCR, rhPCR, and RT-PCR) and ligase chain reaction (LCR) or isothermal PCR reaction may be used to amplify nucleic acid sequences directly from genomic material. For example, the PCR assay may include a number of reagents and components, including a master mix and nucleic acid dye or intercalating agent. In some embodiments, an exemplary PCR master mix may contain template genomic material, such as DNA or RNA, RNase H2 enzyme, RNase H2 enzyme buffer, PCR primers or labeled PCR primers, probes salts such as MgCl₂, a polymerase enzyme, and deoxyribonucleotides. One of skill in the art will be able to identify useful components of a master mix in accordance with the present invention.

### Specific Nucleic Acids

Disclosed herein are specific primers for amplifying the nucleic acid target from a shrimp sample. For example, when the target microorganism is Pacific white shrimp, (Litopenaeus vannamei), either the primer pair SEQ ID NOS: 1 and 2 can be used, or the primer pair SEQ ID NO: 3 and 4 can be used (sequences are found below in Example 1). Or, in another example, a multiplex assay can be carried out which makes use of other sets of primers (SEQ ID NOS: 1 and 2, and SEQ ID NOS: 3 and 4). Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 1, 2, 3, and/or 4. For example, disclosed herein are sequences which consist exactly of any one or a combination of any of SEQ ID NOS: 1-4. Also disclosed are sequences which are identical to any of SEQ ID NOS: 1-4, but which comprise additional nucleic acid on either the 5′ end, the 3′ end, or both ends.

Also disclosed herein are specific primers for amplifying the nucleic acid target from a scallop sample. For example, when the target microorganism is American Scallop (Placopecten magellanicus), either the primer pair SEQ ID NOS: 5 and 6 can be used, or the primer pair SEQ ID NO: 7 and 8 can be used (sequences are found below in Example 1). Or, in another example, a multiplex assay can be carried out which makes use of other sets of primers (SEQ ID NOS: 5 and 6, and SEQ ID NOS: 7 and 8). Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 5, 6, 7, and/or 8. For example, disclosed herein are sequences which consist exactly of any one or a combination of any of SEQ ID NOS: 5-8. Also disclosed are sequences which are identical to any of SEQ ID NOS: 5-8, but which comprise additional nucleic acid on either the 5′ end, the 3′ end, or both ends.

Also disclosed herein are specific primers for amplifying the nucleic acid target from a black grouper sample. For example, when the target microorganism is Black Grouper (Mycteroperca bonaci), either the primer pair SEQ ID NOS: 9 and 10 can be used, or the primer pair SEQ ID NO: 11 and 12 can be used (sequences are found below in Example 3). Or, in another example, a multiplex assay can be carried out which makes use of other sets of primers (SEQ ID NOS: 9 and 10, and SEQ ID NOS: 11 and 12). Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 9, 10, 11, and 12. For example, disclosed herein are sequences which consist exactly of any one or a combination of any of SEQ ID NOS: 9-12. Also disclosed are sequences which are identical to any of SEQ ID NOS: 9-12, but which comprise additional nucleic acid on either the 5′ end, the 3′ end, or both ends.

Also disclosed herein are specific primers for amplifying the nucleic acid target from a black tiger shrimp sample. For example, when the target microorganism is Black Tiger Shrimp (Penaeus monodon), either the primer pair SEQ ID NOS: 13 and 14 can be used, or the primer pair SEQ ID NO: 15 and 16 can be used (sequences are found below in Example 3). Or, in another example, a multiplex assay can be carried out which makes use of other sets of primers (SEQ ID NOS: 13 and 14, and SEQ ID NOS: 15 and 16). Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 13, 14, 15, and 16. For example, disclosed herein are sequences which consist exactly of any one or a combination of any of SEQ ID NOS: 13-16. Also disclosed are sequences which are identical to any of SEQ ID NOS: 13-16, but which comprise additional nucleic acid on either the 5′ end, the 3′ end, or both ends.

Also disclosed herein are specific primers for amplifying the nucleic acid target from a royal red shrimp sample. For example, when the target microorganism is Royal Red Shrimp (Pleoticus robustus), either the primer pair SEQ ID NOS: 17 and 18 can be used, or the primer pair SEQ ID NO: 19 and 20 can be used (sequences are found below in Example 3). Or, in another example, a multiplex assay can be carried out which makes use of other sets of primers (SEQ ID NOS: 17 and 18, and SEQ ID NOS: 19 and 20). Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 17, 18, 19, and 20. For example, disclosed herein are sequences which consist exactly of any one or a combination of any of SEQ ID NOS: 17-20. Also disclosed are sequences which are identical to any of SEQ ID NOS: 17-20, but which comprise additional nucleic acid on either the 5′ end, the 3′ end, or both ends.

Also disclosed herein are specific primers for amplifying the nucleic acid target from a yellowfin snapper sample. For example, when the target microorganism is Yellowfin Snapper (Ocyurus chrysurus), either the primer pair SEQ ID NOS: 21 and 22 can be used, or the primer pair SEQ ID NO: 23 and 24 can be used (sequences are found below in Example 3). Or, in another example, a multiplex assay can be carried out which makes use of other sets of primers (SEQ ID NOS: 21 and 22, and SEQ ID NOS: 23 and 24). Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 21, 22, 23, and 24. For example, disclosed herein are sequences which consist exactly of any one or a combination of any of SEQ ID NOS: 21-24. Also disclosed are sequences which are identical to any of SEQ ID NOS: 21-24, but which comprise additional nucleic acid on either the 5′ end, the 3′ end, or both ends.

Also disclosed herein are specific primers for amplifying the nucleic acid target from a red grouper sample. For example, when the target microorganism is Red Grouper (Epinephelus morio), either the primer pair SEQ ID NOS: 25 and 26 can be used, or the primer pair SEQ ID NO: 27 and 28 can be used, or the primer pair SEQ ID NO: 29 and 30 can be used, or the primer pair SEQ ID NO: 31 and 32 can be used, or the primer pair SEQ ID NO: 33 and 34 can be used, or the primer pair SEQ ID NO: 35 and 36 can be used (sequences are found below in Example 4). Or, in another example, a multiplex assay can be carried out which makes use of multiple sets of primers at the same time, such as SEQ ID NOS: 25 and 26, SEQ ID NOS: 27 and 28, SEQ ID NOS: 29 and 30, SEQ ID NOS: 31 and 32, SEQ ID NOS: 33 and 34, and/or SEQ ID NOS: 35 and 36 can be used in any combination or permutation thereof. Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 25-36. For example, disclosed herein are sequences which consist exactly of any one or a combination of any of SEQ ID NOS: 25-36. Also disclosed are sequences which are identical to any of SEQ ID NOS: 25-36, but which comprise additional nucleic acid on either the 5′ end, the 3′ end, or both ends.

Also disclosed herein are specific primers for amplifying the nucleic acid target from a red drum sample. For example, when the target microorganism is Red Drum (Sciaenops ocellatus), either the primer pair SEQ ID NOS: 37 and 38 can be used, or the primer pair SEQ ID NO: 39 and 40 can be used, or the primer pair SEQ ID NO: 41 and 42 can be used, or the primer pair SEQ ID NO: 43 and 44 can be used, or the primer pair SEQ ID NO: 45 and 46 can be used, or the primer pair SEQ ID NO: 47 and 48 can be used (sequences are found below in Example 4). Or, in another example, a multiplex assay can be carried out which makes use of multiple sets of primers at the same time, such as SEQ ID NOS: 37 and 38, SEQ ID NO: 39 and 40, SEQ ID NO: 41 and 42, SEQ ID NO: 43 and 44, SEQ ID NO: 45 and 46, and/or the SEQ ID NO: 47 and 48 can be used in any combination or permutation thereof. Therefore, disclosed are primers with 80%, 81%, 82%, 83%, 84%, 85%, 86%, 87%, 88%, 89%, 90%, 91%, 92%, 93%, 94%, 95%, 96%, 97%, 98%, 99%, or 100% identical to any of SEQ ID NOS: 37-48. For example, disclosed herein are sequences which consist exactly of any one or a combination of any of SEQ ID NOS: 37-48. Also disclosed are sequences which are identical to any of SEQ ID NOS: 37-48, but which comprise additional nucleic acid on either the 5′ end, the 3′ end, or both ends.

### Kits

Also disclosed herein is a kit for amplification of nucleic acids. Kits may also include additional reagents, e.g., PCR components, such as salts including MgCl₂, a polymerase enzyme, and deoxyribonucleotides, and the like, reagents for DNA or RNA isolation, or enrichment of a biological sample, including for example media such as water, or the like, as described herein. Such reagents or components are well known in the art. Where appropriate, reagents included with such a kit may be provided either in the same container or media as the primer pair or multiple primer pairs, or may alternatively be placed in a second or additional distinct container into which the additional composition or reagents may be placed and suitably aliquoted. Alternatively, reagents may be provided in a single container means.

Specifically, the kit can comprise a container for amplification of a sample; an instrument for sample collection; reagents for amplification of the sample; an instrument for rapid amplification of the sample; and a means of detecting whether the specific food product is present or not. In some embodiments, the kit can further comprise a centrifugation means, as described above, such as a small footprint microcentrifuge. The sample collection instrument can be anything useful in collecting a sample, such as tweezers, a toothpick, or a swab, as described above. the PCR machine for rapid amplification can be a small footprint machine, such as a Watson PCR Machine. Further details are provided above. The reagents can include primers, such as the primers described herein.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: An RHPCR Coupled Lateral Flow Assay for the Specific and on-Site Detection of Pacific White Shrimp and American Scallop

One of the major challenges of developing a species-specific PCR-based assay for the identification of seafood specimens is the close sequence similarity with other phylogenetically related species. The COI gene for target species and species used for their substitution can have more than 95% sequence similarity. Thus, making the assay development process very challenging. Furthermore, the seafood industry prefers the use of simple low-cost equipment, which necessitates the development of a novel diagnostic workflow. A diagnostic workflow capable of performing SNP-level identification, that can fit in a lab-in-a-suitcase-like setup, can be performed at a processing or wholesaler facility using low-cost equipment was needed.

Disclosed herein is a diagnostic workflow where target DNA was amplified using a rhPCR approach and detected using lateral-flow strips. The rhPCR approach uses an RNase H2 enzyme which identifies the target-specific SNP marker, makes an SNP-specific cut, which activates the blocked rhPCR primer, facilitating PCR amplification. If the test sample lacks the target SNP marker, the block rhPCR primer is not cleaved by the RNase H2 enzyme, resulting in no amplification. The rhPCR was performed using a portable conventional PCR, enabling the use of low-cost equipment. Additionally, in the standardized workflow, the rhPCR primers were modified with FAM/Biotin or Digoxigenin labels, which enabled the convenient detection of two amplicons (i.e., target and IAC) on lateral flow strips.

TABLE 1
Primers for the detection of Pacific
white shrimp (Litopenaeus vannamei)

| | Sequence | Amplicon |
|---|---|---|
| Rh-L. | /56-FAM/GAATGGTTGAAAG | 92 bp |
| vannamei- | AGGTGTrCGGAAA/3SpC3/ | |
| 297F | (SEQ ID NO: 1) | |
| Rh-L. | /5BiosG/CCAAGATCTACTG | |
| vannamei- | AAGCTCCrAGCGTT/3SpC3/ | |
| 388R | (SEQ ID NO: 2) | |
| FAM_IAC- | /56-FAM/CGCAACGATGAT | 85 bp |
| 2F | TATTTTCT (SEQ ID NO: 3) | |
| DIG_IAC- | /5DiGN/GTNCCTACTATTC | |
| 86R | CAGCTCA (SEQ ID NO: 4) | |

The DNA samples previously isolated from tissue samples were used for the assay standardization. The rhPCR assay was performed using a 2×KAPA Force master mix (Kapa Biosystems) with 20 mU of RNase H2 Enzyme. The assay used the following PCR program: Initial Denaturation at 98° C. for 300 sec, followed by 35 cycles of denaturation at 95° C. for 10 sec, annealing at 56° C. for 30 sec, and extension at 72° C. for 30 sec. Followed by a final extension at 72° C. for 300 sec. The amplicons generated in the duplex PCR reaction were detected using the HybriDetect 2T (milenia biotech). The Litopenaeus vannamei samples formed two bands (target and IAC bands) on the lateral flow test strips, whereas other species formed one band (IAC band) on the lateral flow strips.

TABLE 2
Primers for the identification of Atlantic
Scallop (Placopecten magellanicus)

| | Sequence | Amplicon |
|---|---|---|
| Rh.PM-2F | /56-FAM/GGGTTTGGGAATT | 250 bp |
| | GGYTGCTrCCCTTG/3SpC3/ | |
| | (SEQ ID NO: 5) | |
| Rh.PM-2R | /5BiosG/CAGCAGAAGACCT | |
| | TACCCCArGCCAAG/3SpC3/ | |
| | (SEQ ID NO: 6) | |
| FAM_ | /56-FAM/CCTCTTGCCATCG | 99 bp |
| 16SRna-F | GATGTG | |
| | (SEQ ID NO: 7) | |
| DIG_ | /5DiGN/GGCTGGTCATCCTC | |
| 16SRna-R | TCAGACC (SEQ ID NO: 8) | |

The DNA samples previously isolated from tissue samples were used for the assay standardization. The rhPCR assay was performed using a 2×KAPA Force master mix with 20 mU of RNase H2 Enzyme. A novel approach was used for the standardization of IAC for scallop assay. A conserved primer pair targeting the bacterial 16S rRNA gene sequence was used. The novelty of this IAC approach is food samples have some levels of bacterial load, which will amplify generating an IAC band. If needed a diluted bacterial DNA (1 ng/reaction) can be added to the reaction mix.

The scallop and the bacterial 16S rRNA gene primer pair independently amplified their respective targets in the duplex reaction without any coemption. The assay used the following PCR program: initial denaturation at 98° C. for 300 seconds, followed by 35 cycles of denaturation at 95° C. for 10 seconds, annealing at 60° C. for 30 sec, and extension at 72° C. for 30 seconds. Followed by a final extension at 72° C. for 300 seconds. The amplicons generated in the duplex PCR reaction were detected using the HybriDetect 2T (Milenia biotech). The assay showed specific amplification for the American Scallop (Placopecten magellanicus) samples.

### Example 2: Litopenaeus Vannamei Lateral Flow Assay Procedure

Place the sample in a sterile sample bag.
Crush the sample by placing pressure with your palm or thumb.
Take the smallest amount of shrimp tissue using a disposable toothpick. (The smaller the sample size, the better it is).
Transfer the shrimp tissue to 100 μL of extraction reagent (premeasured).
Place the sample in a PCR instrument or a dry bath and heat at 95° C. for 30 minutes.
Allow the sample to cool down for 5 minutes.
Centrifuge the samples at 5000×g for 5 minutes.
Transfer two microliters of the supernatant (avoid any tissue debris) into a 18 μl nuclease-free water tube (premeasured) and vortex to mix the sample.
Use 2 μl of the diluted DNA samples in the PCR reaction tube (premeasured).
PCR Condition:
Use the following PCR Program:

| | | |
|---|---|---|
| 98 C. | 300 | s |
| 95 C. | 10 | s |
| 59 C. | 30 | s |
| 72 C. | 30 | s |
| 72 C. | 300 | s |

Add 15 μl of PCR product and add it to the lateral flow reagent (premeasured). Mix the sample and add a lateral flow stick.
Incubate the sample for 5 minutes.
Record the results: Three bands on the lateral flow stick indicate the sample was Vannamei shrimp. The presence of one or two bands on the lateral flow stick indicates the sample was not a vannamei shrimp.

### Example 3: Primers for Black Grouper, Black Tiger Shrimp, Royal Red Shrimp, and Yellowtail Snapper

| Species | | |
|---|---|---|
| Black Grouper | 83-F | GGAGCCCTACTAGGCGAT (SEQ ID NO: 9) |
| | 83-Rh-F | GGAGCCCTACTAGGCGArUGATCC/3SpC3/ |
| | | (SEQ ID NO: 10) |
| | 83-FAM-Rh-F | /56- |
| | | FAM/GGAGCCCTACTAGGCGArUGATCC/3 |
| | | SpC3/ (SEQ ID NO: 10 plus FAM) |
| | 310-R | GTTCAACCAGTACCGGCTCCA (SEQ ID |
| | | NO: 11) |
| | 310-Rh-R | GTTCAACCAGTACCGGCTCCrAGCTTA/3Sp |
| | | C3/ (SEQ ID NO: 12) |
| | 310-BIO-Rh-R | /5BiosG/GTTCAACCAGTACCGGCTCCrAGC |
| | | TTA/3SpC3/ (SEQ ID NO: 12 plus FAM) |
| Black Tiger | 179-F | GGTTTCGGGAATTGGCTT (SEQ ID NO: 13) |
| Shrimp | | |
| | 179-Rh-F | GGTTTCGGGAATTGGCTrUGTCCAA/3SpC3/ |
| | | (SEQ ID NO: 14) |
| | 179-FAM-Rh-F | /56- |
| | | FAM/GGTTTCGGGAATTGGCTrUGTCCAA/ |
| | | 3SpC3/ (SEQ ID NO: 14 plus FAM) |
| | 304-R | GTTCCCACTCCTCTTTCG (SEQ ID NO: 15) |
| | 304-Rh-R | GTTCCCACTCCTCTTTCrGACTAC/3SpC3/ |
| | | (SEQ ID NO: 16) |
| | 304-BIO-Rh-R | /5BiosG/GTTCCCACTCCTCTTTCrGACTAC/ |
| | | 3SpC3/ (SEQ ID NO: 16 plus FAM) |
| Royal Red | 403-F | CCTTGCTGGGGTTTCTTCAA |
| Shrimp | | (SEQ ID NO: 17) |
| | 403-Rh-F | CCTTGCTGGGGTTTCTTCAArUTTTAT/3Sp |
| | | C3/ (SEQ ID NO: 18) |
| | 403-FAM-Rh-F | /56- |
| | | FAM/CCTTGCTGGGGTTTCTTCAArUTTTA |
| | | T/3SpC3/ (SEQ ID NO: 18 plus FAM) |
| | 564-R | GCTCCCGCTAAGACTGGTAA (SEQ ID NO: |
| | | 19) |
| | 564-Rh-R | GCTCCCGCTAAGACTGGTAArGGATAG/3S |
| | | pC3/ (SEQ ID NO: 20) |
| | 564-BIO-Rh-R | /5BiosG/GCTCCCGCTAAGACTGGTAArGGA |
| | | TAG/3SpC3/ (SEQ ID NO: 20 plus FAM) |
| Yellowtail | 478-F | CATGAAACCTCCTGCCATT (SEQ ID NO: |
| Snapper | | 21) |
| | 478-Rh-F | CATGAAACCTCCTGCCATrUTCCCT/3SpC3/ |
| | | (SEQ ID NO: 22) |
| | 478-FAM-Rh-F | /56- |
| | | FAM/CATGAAACCTCCTGCCATT/3SpC3/ |
| | | (SEQ ID NO: 22 plus FAM) |
| | 574-R | AGATTTCGGTCTGTAAGAAGC (SEQ ID |
| | | NO: 23) |
| | 574-Rh-R | AGATTTCGGTCTGTAAGAAGrCattgC/3SpC3 |
| | | / (SEQ ID NO: 24) |
| | 574-BIO-Rh-R | /5BiosG/AGATTTCGGTCTGTAAGAAGrCattg |
| | | C/3SpC3/ ((SEQ ID NO: 24 plus FAM) |

### Example 4, Red Grouper and Red Drum Primers

| | | |
|---|---|---|
| Red | 55-F | GCTTATTCGAGCTGAGCTG (SEQ ID NO: 25) |
| Grouper | | |
| | 55-Rh-F | GCTTATTCGAGCTGAGCTrGagccG/3SpC3/ (SEQ ID |
| | | NO: 26) |
| | 55-FAM-Rh- | /56-FAM/GCTTATTCGAGCTGAGCTrGagccG/3SpC3/ |
| | F | (SEQ ID NO: 26 plus FAM) |
| | 72-F | TGAGCCAACCTGGGGCTT (SEQ ID NO: 27) |
| | 72-Rh-F | TGAGCCAACCTGGGGCTrUtgctG/3SpC3/ (SEQ ID NO: |
| | | 28) |
| | 72-FAM-Rh- | /56-FAM/TGAGCCAACCTGGGGCTrUtgctG/3SpC3/ (SEQ |
| | F | ID NO: 28 plus FAM) |
| | 128-F | GCGCATGCATTTGTAATAATC (SEQ ID NO: 29) |
| | 128-Rh-F | GCGCATGCATTTGTAATAATrCttttG/3SpC3/ (SEQ ID |
| | | NO: 30) |
| | 128-FAM- | /56-FAM/GCGCATGCATTTGTAATAATrCttttG/3SpC3/ |
| | Rh-F | (SEQ ID NO: 30 plus FAM) |
| | 278-R | CGAAGCTAAGAGAAGCAA (SEQ ID NO: 31) |
| | 278-Rh-R | CGAAGCTAAGAGAAGCArAgaaaT/3SpC3/ (SEQ ID NO: |
| | | 32) |
| | 278-FAM- | /5BiosG/CGAAGCTAAGAGAAGCArAgaaaT/3SpC3/ (SEQ |
| | Rh-R | ID NO: 32 plus FAM) |
| | 292-R | GCTTCAACTCCAGACGAA (SEQ ID NO: 33) |
| | 292-Rh-R | GCTTCAACTCCAGACGArAgctaC/3SpC3/ (SEQ ID NO: |
| | | 34) |
| | 292-FAM- | /5BiosG/GCTTCAACTCCAGACGArAgctaC/3SpC3/ (SEQ |
| | Rh-R | ID NO: 34 plus 5BiosG) |
| | 304-R | GTACCAGCACCGGCTTCA (SEQ ID NO: 35) |
| | 304-Rh-R | GTACCAGCACCGGCTTCrAactcT/3SpC3/ (SEQ ID NO: |
| | | 36) |
| | 304-FAM- | /5BiosG/GTACCAGCACCGGCTTCrAactcT/3SpC3/ (SEQ |
| | Rh-R | ID NO: 36 plus 5BiosG) |
| Red | 29-F | GGAATAGTAGGCACAGCCT (SEQ ID NO: 37) |
| Drum | | |
| | 29-Rh-F | GGAATAGTAGGCACAGCCrUtaagA/3SpC3/ (SEQ ID NO: |
| | | 38) |
| | 29-FAM-Rh- | /56-FAM/GGAATAGTAGGCACAGCCrUtaagA/3SpC3/ |
| | F | (SEQ ID NO: 38 plus FAM) |
| | 227-F | GCATTCCCCCGAATAAATAAT (SEQ ID NO: 39) |
| | 227-Rh-F | GCATTCCCCCGAATAAATAArUatgaT/3SpC3/ (SEQ ID |
| | | NO: 40) |
| | 227-FAM- | /56-FAM/GCATTCCCCCGAATAAATAArUatgaT/3SpC3/ |
| | Rh-F | (SEQ ID NO: 40 plus FAM) |
| | 358-F | TGCACACGCAGGAGCTTCC (SEQ ID NO: 41) |
| | 358-Rh-F | TGCACACGCAGGAGCTTCCgtcgT/3SpC3/ (SEQ ID NO: |
| | | 42) |
| | 358-FAM- | /56-FAM/TGCACACGCAGGAGCTTCCgtcgT/3SpC3/ |
| | Rh-F | (SEQ ID NO: 42 plus FAM) |
| | 283-R | ACCTGAGGAGGTAAGGAGA (SEQ ID NO: 43) |
| | 283-Rh-R | ACCTGAGGAGGTAAGGAGrAagaaT/3SpC3/ (SEQ ID |
| | | NO: 44) |
| | 283-FAM- | /5BiosG/ACCTGAGGAGGTAAGGAGrAagaaT/3SpC3/ |
| | Rh-R | (SEQ ID NO: 44 plus 5BiosG) |
| | 376-R | AAAGATGGCTAAGTCGACG (SEQ ID NO: 45) |
| | 376-Rh-R | AAAGATGGCTAAGTCGACrGgaagT/3SpC3/ (SEQ ID NO: |
| | | 46) |
| | 376-FAM- | /5BiosG/AAAGATGGCTAAGTCGACrGgaagT/3SpC3/ |
| | Rh-R | (SEQ ID NO: 46 plus 5BiosG) |
| | 475-R | GGTGTCTGATACTGGGAAATA (SEQ ID NO: 47) |
| | 475-Rh-R | GGTGTCTGATACTGGGAAATrAgcggA/3SpC3/ (SEQ ID |
| | | NO: 48) |
| | 475-FAM- | /5BiosG/GGTGTCTGATACTGGGAAATrAgcggA/3SpC3/ |
| | Rh-R | (SEQ ID NO: 48 plus 5BiosG) |

### REFERENCES

1. Bayha, K. M., Graham, W. M., & Hernandez Jr, F. J. (2008). Multiplex assay to identify eggs of three fish species from the northern Gulf of Mexico, using locked nucleic acid Taqman real-time PCR probes. Aquatic Biology, 4(1), 65-73. https://doi.org/10.3354/ab00100
2. Bradley, E., & Bryan, C. E. (1975). Life history and fishery of the Red Snapper (Lutjanus campechanus) in the northwestern Gulf of Mexico 1970-1974.
3. Chou, Q., Russell, M., Birch, D. E., Raymond, J., & Bloch, W. (1992). Prevention of pre-PCR mis-priming and primer dimerization improves low-copy-number amplifications. Nucleic acids research, 20(7), 1717-1723. https://doi.org/10.1093/nar/20.7.1717
4. Crissman, J., Lin, Y., Separa, K., Duquette, M., Cohen, M., Velasquez, C., & Cujec, T. (2020). RNase H-dependent PCR enables highly specific amplification of antibody variable domains from single B-cells. PloS one, 15(11), e0241803. https://doi.org/10.1371/journal.pone.0241803
5. Dawan, J., & Ahn, J. (2022). Application of DNA barcoding for ensuring food safety and quality. Food Science Biotechnology 31, 1355-1364 https://doi.org/10.1007/s10068-022-01143-7
6. Frigerio, J., Gorini, T., Palumbo, C. et al. (2023). A Fast and Simple DNA Mini-barcoding and RPA Assay Coupled with Lateral Flow Assay for Fresh and Canned Mackerel Authentication. Food Anal. Methods 16, 426-435. https://doi.org/10.1007/s12161-022-02429-6
7. Gomes, G., Schneider, H., Vallinoto, M., Santos, S., Orti, G., & Sampaio, I. (2008). Can Lutjanus purpureus (South red snapper) be “legally” considered a red snapper (Lutjanus campechanus)? Genetics and Molecular Biology, 31, 372-376. https://doi.org/10.1590/S1415-47572008000200035
8. Hoorfar, J., Malorny, B., Abdulmawjood, A., Cook, N., Wagner, M., & Fach, P. (2004). Practical considerations in design of internal amplification controls for diagnostic PCR assays. Journal of clinical microbiology, 42(5), 1863-1868. https://doi: 10.1128/jcm.42.5.1863-1868.2004
9. https://www.fisheries.noaa.gov/species/red-snapper
10. Hu, Y., Huang, S. Y., Hanner, R., Levin, J. & Lu, X. Study of fish products in metro Vancouver using DNA barcoding methods reveals fraudulent labeling. Food Control 94, 38-47 (2018).
11. Huang, T. S., Marshall, M. R., & Wei, C. I. (1995). Identification of red snapper (Lutjanus campechanus) using electrophoretic techniques. Journal of Food Science, 60(2), 279-283.
12. Isaacs, R. B., & Hellberg, R. S. (2020). Authentication of red snapper (Lutjanus campechanus) fillets using a combination of real-time PCR and DNA barcoding. Food Control, 118, 107375. https://doi.org/10.1016/j.foodcont.2020.107375
13. Ivanova, N. V., Zemlak, T. S., Hanner, R. H., & Hebert, P. D. (2007). Universal primer cocktails for fish DNA barcoding. Molecular Ecology Notes, 7(4), 544-548.
14. Kim, J. H., & Oh, S. W. (2021). A colorimetric lateral flow assay based on multiplex PCR for the rapid detection of viable Escherichia coli O157:H7 and Salmonella Typhimurium without enrichment. LWT, 152, 112242. doi: 10.1016/j.lwt.2021.112242
15. Kwawukume, S., Velez, F. J., Williams, D., Cui, L., & Singh, P. (2023). Rapid PCR-lateral flow assay for the onsite detection of Atlantic White Shrimp. Food Chemistry: Molecular Sciences, 100164.
16. Lee, Y.-M., Lee, S., & Kim, H.-Y. (2021). A Multiplex PCR Assay Combined with Capillary Electrophoresis for the Simultaneous Identification of Atlantic Cod, Pacific Cod, Blue Whiting, Haddock, and Alaska Pollock. Foods, 10(11), 2631. MDPI AG. Retrieved from http://dx.doi.org/10.3390/foods10112631
17. Logan, C. A., Alter, S. E., Haupt, A. J., Tomalty, K., & Palumbi, S. R. (2008). An impediment to consumer choice: overfished species are sold as Pacific red snapper. Biological Conservation, 141(6), 1591-1599.
18. National Marine Fisheries Service (2022). Fisheries of the United States, 2020. U.S. Department of Commerce, NOAA Current Fishery Statistics No. 2020. Available at: https://www.fisheries.noaa.gov/national/sustainable-fisheries/fisheries-united-states
19. Palumbi, S. R. (1996). Nucleic acids II: the polymerase chain reaction. In ‘Molecular Systematics’. (Eds DM Hillis, C. Moritz and BK Mable.) pp. 205-247.
20. https://sequencing.roche.com/us/en/products/group/kapa-probe-force-qpcr-kits.html
21. Shen, K.; Hu, X.; Sun, L.; Han, C.; Yang, J. (2022). Development of a Lateral Flow Strip with a Positive Readout for the Onsite Detection of Aflatoxin B1. Molecules 2022, 27, 4949. https://doi.org/10.3390/molecules27154949
22. Single Laboratory Validated Method for DNA-Barcoding for the Species Identification of Fish | FDA
23. Spencer, E. T., & Bruno, J. F. (2019). Fishy business: red snapper mislabeling along the coastline of the Southeastern United States. Frontiers in Marine Science, 6, 513. https://doi.org/10.3389/fmars.2019.00513
24. Teo, I. A., Choi, J. W., Morlese, J., Taylor, G., & Shaunak, S. (2002). LightCycler qPCR optimisation for low copy number target DNA. Journal of immunological methods, 270(1), 119-133. https://doi.org/10.1016/s0022-1759(02)00218-1
25. Velez, F. J., Bosilevac, J. M., Delannoy, S., Fach, P., Nagpal, R., & Singh, P. (2022). Development and validation of high-resolution melting assays for the detection of potentially virulent strains of Escherichia coli O103 and O121. Food Control, 139, 109095. https://doi.org/10.1016/j.foodcont.2022.109095
26. Velez, F. J., Bosilevac, J. M., Mishra, A., & Singh, P. (2023). Universal hydrolysis probe-based approach for specific detection and genotyping of foodborne pathogens. Journal of Microbiological Methods, 204, 106632. https://doi.org/10.1016/j.mimet.2022.106632
27. Warner, K., Timme, W., Lowell, B., & Hirshfield, M. (2013). Oceana Study Reveals Seafood Fraud Nationwide. Accessed Nov. 9, 2022. Retrieved from https://oceana.org/reports/oceana-study-reveals-seafood-fraud-nationwide/28.
28. Wells, R. D., Cowan Jr, J. H., & Fry, B. (2008). Feeding ecology of red snapper Lutjanus campechanus in the northern Gulf of Mexico. Marine Ecology Progress Series, 361, 213-225. https://doi.org/10.3354/meps07425
29. Will, S. G. (1999). U.S. Pat. No. 6,001,611. Washington, DC: U.S. Patent and Trademark Office. Accessed from https://patents.google.com/patent/U.S. Pat. No. 6,001,611A/en on Feb. 9, 2023
30. Zhao L, Wang J, Chen M, et al (2022) Development and application of recombinase polymerase amplification assays for rapid detection of Escherichia coli O157 in food. Food Analytical Methods 1-8 https://doi.org/10.1007/s12161-022-02250-1

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

What is claimed is:

1. A method of rapidly determining if a specific food product is present or not, the method comprising:
a. providing a sample comprising at least one target sequence;
b. placing the sample into at least one container;
c. using reagents to amplify a sample;
d. amplifying a sample using a small footprint amplification device, wherein the sample is amplified by exposing it to different sets of primers in conditions suitable for nucleic acid amplification, where each set of primers comprises of a forward and reverse primer, wherein the primers comprise 90% or more identity to at least one set primers comprising SEQ ID NOS: 1 and 2; or SEQ ID NOS: 3 and 4; or SEQ ID NOS: 5 and 6; or SEQ ID NOS: 7 and 8; or SEQ ID NOS: 9 and 10; or SEQ ID NOS: 11 and 12; or SEQ ID NOS: 13 and 14; or SEQ ID NOS: 15 and 16; or SEQ ID NOS: 17 and 18; or SEQ ID NOS: 19 and 20; or SEQ ID NOS: 21 and 22; or SEQ ID NOS: 23 and 24; or SEQ ID NOS: 25 and 26; or SEQ ID NOS: 27 and 28; or SEQ ID NOS: 29 and 30; or SEQ ID NOS: 31 and 32; or SEQ ID NOS: 33 and 34; or SEQ ID NOS: 35 and 36; or SEQ ID NOS: 37 and 38; or SEQ ID NOS: 39 and 40; or SEQ ID NOS: 41 and 42; or SEQ ID NOS: 43 and 44; or SEQ ID NOS: 45 and 46; or SEQ ID NOS: 47 and 48;
e. exposing the amplified sequence to a means of detection, wherein the means of detection provides a present/not present result; and
f. identifying whether the food product is present or not based on the results of step e).

2. The method of claim 1 wherein the food product is fish or shellfish.

3. The method of claim 2, wherein the method discriminates between Atlantic white shrimp (Litopenaeus setiferus) and Pacific white shrimp (Litopenaeus vannamei).

4. The method of claim 2, wherein the method discriminates between American Scallop (Placopecten magellanicus) and other scallop species.

5. The method of claim 1, wherein the sample is amplified using a Watson PCR machine.

6. The method of claim 1, wherein the reagents include:
a. PCR master mix;
b. Taq polymerase;
c. Primers or rhPCR primer; and
d. RNase H2 enzyme, RNase H2 enzyme buffer

7. The method of claim 1, wherein the Taq polymerase or the master mix has an inhibitor resistant properties.

8. The method of claim 1, wherein the detection means comprises a lateral flow assay.

9. The method of claim 1, wherein the detection means comprises a qPCR machine.

10. The method of claim 1, wherein the sample is centrifuged prior to the amplification step.

11. The method of claim 1, wherein the centrifuge is a small footprint microcentrifuge.

12. The method of claim 6, wherein the reagents are premixed.

13. The method of claim 12, wherein the premixed reagents are activated by PCR grade water or RNase H2 enzyme with reaction buffer.

14. A kit for rapidly determining if a specific food product is present or not, wherein the kit comprises:
a. a container for amplification of a sample;
b. an instrument for sample collection;
c. reagents for amplification of the sample, including primers comprising 90% or more identity to at least one set primers comprising SEQ ID NOS: 1 and 2; or SEQ ID NOS: 3 and 4; or SEQ ID NOS: 5 and 6; or SEQ ID NOS: 7 and 8; or SEQ ID NOS: 9 and 10; or SEQ ID NOS: 11 and 12; or SEQ ID NOS: 13 and 14; or SEQ ID NOS: 15 and 16; or SEQ ID NOS: 17 and 18; or SEQ ID NOS: 19 and 20; or SEQ ID NOS: 21 and 22; or SEQ ID NOS: 23 and 24; or SEQ ID NOS: 25 and 26; or SEQ ID NOS: 27 and 28; or SEQ ID NOS: 29 and 30; or SEQ ID NOS: 31 and 32; or SEQ ID NOS: 33 and 34; or SEQ ID NOS: 35 and 36; or SEQ ID NOS: 37 and 38; or SEQ ID NOS: 39 and 40; or SEQ ID NOS: 41 and 42; or SEQ ID NOS: 43 and 44; or SEQ ID NOS: 45 and 46; or SEQ ID NOS: 47 and 48;
d. an instrument for rapid amplification of the sample;
e. a means of detecting whether the specific food product is present or not.

15. The kit of claim 14, wherein the kit further comprises a centrifugation means.

16. The kit of claim 15, wherein the centrifugation means is a small footprint microcentrifuge.

17. The kit of claim 14 wherein the instrument for sample collection is tweezers.

18. The kit of claim 14, wherein the instrument for rapid amplification is a small footprint PCR machine.

19. The kit of claim 18, wherein the instrument for amplification is a Watson PCR machine.

20. A nucleic acid with 90% or more identity to SEQ ID NOS: 1-48.
